(19) United States
(12) Patent Application Publication
(10) Pub. No.: US 2026/0060636 A1
(43) Pub. Date: Mar. 5, 2026

(54) ANALYSING HEART OR RESPIRATORY-SYSTEM SOUNDS

(71) Applicants: Universitetet i Tromsø - Norges Arktiske Universitet, Tromsø (NO); Medsensio AS, Tromsø (NO); Universitetet I Oslo, Oslo (NO)

(72) Inventors: LARS AILO ASLAKSEN BONGO, Tromsø (NO); PER NIKLAS BENZLER WAALER, Tromsø (NO); HASSE MELBYE, Tromsø (NO); MARKUS KREUTZER JOHNSEN, Tromsø (NO); JOHAN FREDRIK RAVN, Tromsø (NO); HENRIK SCHIRMER, Tromsø (NO); JUAN CARLOS AVILES SOLIS, Tromsø (NO)

(21) Appl. No.: 19/104,467

(22) PCT Filed: Aug. 18, 2023
(86) PCT No.: PCT/EP2023/072820

§ 371 Date: Feb. 18, 2025

(30) Foreign Application Priority Data
Aug. 18, 2022 (GB) 2212073.7

(51) Int. Cl. A61B 7/04; A61B 5/00; A61B 7/00

(52) U.S. Cl. CPC A61B 7/04; A61B 5/7264; A61B 7/003

(57) ABSTRACT

A method, apparatus and computer software for determining rate estimates from sounds emanating from a heart or respiratory system of a human or animal body. A plurality of sound recordings of a heart or respiratory system of a human or animal body are received, wherein each sound recording is or has been captured by a microphone positioned at a respective location on the exterior of the human or animal body. For each of the sound recordings, a respective individual rate is determined (51) by analysing a respective autocorrelation function of the sound recording and determining a respective quality measure. An aggregate rate estimate for the heart or respiratory system is determined (53) by evaluating a weighted combination of two or more of the plurality of individual rate estimates, wherein each of the individual rate estimates included in the weighted combination is weighted at least in part by the respective quality measure for the respective sound recording.

### BACKGROUND OF THE INVENTION

This invention relates to methods and apparatus for determining rate estimates from sounds emanating from the heart or respiratory system of humans or animals.

Many sounds produced by the body, in particular produced by the heart and by the respiratory system, are periodic. In the case of the heart, across each heart beat period, different physiological phases of the heart's movement (e.g. diastole and systole phases) can be identified by analysing the sound produced. Similarly, for a breath cycle, a phase of inhalation followed by a phase of exhalation can be identified in sound emanating from the lungs. Segmenting sound recordings into phases that indicate different physiological movements can provide valuable information, e.g. for assessing fitness or well-being, or to identify certain abnormalities in the function of the organ.

Segmentation of such recordings can be achieved using a signal processing algorithm executed by a computer program. However, the accuracy of this segmentation process may vary depending on the quality of the sound recording. Segmentation results may be improved by pre-processing the sound recording beforehand. Part of this pre-processing can involve calculating an estimate for the rate of the periodic sound in the recording, for example estimating a heart rate. The accuracy of the heart rate estimate therefore affects the accuracy of the segmentation of the recording. The accuracy of this heart rate estimate can be affected by noise or interference in the sound recording.

Embodiments of the present invention seek to provide a better rate estimate from heart or respiratory-system sounds. This may be useful for improving the accuracy of subsequent processing and analysis of sound recordings, or for other purposes.

### SUMMARY OF THE INVENTION

From a first aspect, the invention provides a method for determining rate estimates from sounds emanating from a heart or respiratory system of a human or animal body, the method comprising:
receiving a plurality of sound recordings of a heart or respiratory system of a human or animal body, wherein each sound recording is or has been captured by a microphone positioned at a respective location on the exterior of the human or animal body;
for each of the plurality of sound recordings, determining a respective individual rate estimate for the sound recording by analysing a respective autocorrelation function of the sound recording and determining a respective quality measure for the sound recording; and
determining an aggregate rate estimate for the heart or respiratory system by evaluating a weighted combination of two or more of the plurality of individual rate estimates, wherein each of the individual rate estimates included in the weighted combination is weighted at least in part by the respective quality measure for the respective sound recording.

From a second aspect, the invention provides an apparatus for determining rate estimates from sounds emanating from a heart or respiratory system of a human or animal body, the apparatus comprising a processing system configured to:
receive a plurality of sound recordings of a heart or respiratory system of a human or animal body, wherein each sound recording is or has been captured by a microphone positioned at a respective location on the exterior of the human or animal body;
for each of the plurality of sound recordings, determine a respective individual rate estimate for the sound recording by analysing a respective autocorrelation function of the sound recording and determine a respective quality measure for the sound recording; and
determine an aggregate rate estimate for the heart or respiratory system by evaluating a weighted combination of two or more of the plurality of individual rate estimates, wherein each of the individual rate estimates included in the weighted combination is weighted at least in part by the respective quality measure for the respective sound recording.

From a third aspect, the invention provides computer software for determining rate estimates from sounds emanating from a heart or respiratory system of a human or animal body, wherein the software comprises instructions which, when executed on a processing system, cause the processing system to:
receive a plurality of sound recordings of a heart or respiratory system of a human or animal body, wherein each sound recording is or has been captured by a microphone positioned at a respective location on the exterior of the human or animal body;
for each of the plurality of sound recordings, determine a respective individual rate estimate for the sound recording by analysing a respective autocorrelation function of the sound recording and determine a respective quality measure for the sound recording; and
determine an aggregate rate estimate for the heart or respiratory system by evaluating a weighted combination of two or more of the plurality of individual rate estimates, wherein each of the individual rate estimates included in the weighted combination is weighted at least in part by the respective quality measure for the respective sound recording.

Thus it will be seen that, in accordance with embodiments of the invention, an aggregate rate estimate is calculated from a plurality of captured sound recordings, which can provide a more robust overall rate estimate than a rate estimate calculated from just a single sound recording. This aggregate rate estimate may then inform any further processing of each sound recording, such that the accuracy of any subsequent processing steps (e.g. segmenting one or more of the sound recordings) can be improved.

The sound recordings may capture sound from one or a pair of lungs or from a windpipe of a human or animal body, but in a set of embodiments each sound recording captures sound from the human heart. The individual and aggregate rate estimates may be heart rate estimates. The aggregate estimate of the heart rate may then be used to inform subsequent processing steps, such as segmenting each sound recording to identify one or more diastole and systole phases in the sound recording. Seeking to provide a better estimate of the heart rate may advantageously improve the accuracy of any such subsequent processing step.

In some embodiments, two or more, or all, of the sound recordings may be captured, or may have been captured, from the same location on the exterior of the human or animal body (e.g. over different time periods).

However, in other embodiments, each sound recording is or has been captured at a different respective locations on the body. This can produce sound recordings of the heart or respiratory system which may show different types of sound profile. In some embodiments, two, three or four sound recordings are received, wherein each sound recording is or has been captured adjacent a different respective heart valve (i.e. a different one of an aortic valve, a pulmonary valve, a tricuspid valve, and a mitral valve).

In a set of embodiments, the sound recordings are captured sequentially, such that each recording is captured over a different non-overlapping period of time. Although different in time, it is beneficial if the recordings are taken close to each other in time, such that the underlying rate estimate (e.g. heart rate in bpm) does not change significantly (e.g. less than 10%) between the sound recordings.

In some embodiments, the same microphone is used for capturing each sound recording, while in other embodiments a plurality of microphones (e.g. transducer elements) may be used.

The sound recordings may be pre-processed before determining the respective autocorrelation function of each sound recording. For example, they may be down-sampled and/or band-pass filtered and/or smoothed. This may help to better reveal the periodicity of each sound recording.

In a set of embodiments, determining each individual rate estimate comprises identifying a primary autocorrelation peak in the respective autocorrelation function, and calculating the individual rate estimate from a time delay of the primary autocorrelation peak. It may comprise identifying the respective primary autocorrelation peak within a respective search interval. The search interval may correspond to maximum and minimum expected time periods within reasonable physiological boundaries. For example, for the heart, the search interval may be set between delays of 0.5s and 2s, which would respectively indicate a heart rate between 30 and 120 bpm.

In a set of embodiments, the primary autocorrelation peak is selected from a set of candidate peaks in the autocorrelation function within the search interval. For example, the three highest peaks within a search interval may be identified, and included in the set of candidate peaks. An initial primary peak may be identified as the tallest of the candidate peaks, or by any other criteria. After selecting an initial primary peak, a unit-fraction search interval may be determined, defining a time interval spanning and/or centred around a predetermined unit fraction (e.g. a half or a third) of the time delay of the initial primary peak.

In a set of embodiments where the unit-fraction search interval is calculated for an initial primary peak, the method may further involve determining whether another of the set of candidate peaks fall within the unit-fraction search interval and has an autocorrelation above a minimum level. The minimum level may be fixed or it may depend on the autocorrelation value of the initial primary peak—e.g. being at least a predetermined fraction thereof. Where such another peak is identified, it may be used for determining the respective individual rate estimate, instead of the initial primary peak. This may advantageously identify where the cycle period (cardiac cycle or breathing cycle) indicated by the initial primary peak is a multiple of the true cycle period, and then discard the initial peak which would otherwise lead to an erroneous rate estimate.

The quality measure for a sound recording may represent a likelihood that the respective heart rate estimate determined for the sound recording is accurate. In a set of embodiments, the quality measure for each sound recording depends at least in part on one or more properties of the respective autocorrelation function of the sound recording. Thus, determining the quality measure may comprise determining a prominence of a primary peak in the autocorrelation function relative to an adjacent region of the respective autocorrelation function. Alternatively, or additionally, it may comprise determining a prominence of a primary peak relative to one or more further peaks of the autocorrelation signal, and/or determining a periodicity of a succession of primary peaks of the autocorrelation function (e.g. spanning several heart beats or breathing cycles). A confidence measure for a sound recording may be calculated in dependence on one of more of these properties. The quality measure for the sound recording may then depend, at least in part, on the respective confidence measure.

Analysing such properties of each autocorrelation function can advantageously mean that a measure of how much confidence may be placed on each individual rate estimate can be determined, from which the quality measures for the sound recordings may be determined. For instance, if the peaks in the autocorrelation signal are less prominent, or do not appear to be periodic over several heart beats or breaths, it is more likely that noise, or any other kind of interference could have affected the autocorrelation, resulting in an incorrect determination of the heartbeat or breathing rate.

The quality measure for a sound recording may additionally depend on one or more properties of the respective autocorrelation functions of every other sound recording in the plurality of sound recordings. It may depend on (e.g. equal) a ratio of a confidence measure for the respective sound recording to the sum of the confidence measures across all of the plurality of sound recordings.

The aggregate rate estimate may be calculated as a weighted sum of the two or more individual rate estimates.

In some embodiments, the aggregate rate estimate may be a weighted combination of all of the plurality of individual rate estimates (i.e. with non-zero weight terms applied to each individual rate estimate). Such an aggregate rate estimate (e.g. h as described below) may represent an average rate determined across all the sound recordings.

However, in some embodiments, the aggregate rate estimate may instead be a weighted combination of only a subset of the plurality of individual rate estimates. For each of the plurality of sound recordings, a respective aggregate rate estimate may be determined as a weighted combination of all of the plurality of individual rate estimates apart from the individual rate estimate determined for that respective sound recording. Thus a respective weighted combination may be calculated by excluding the individual rate estimate of the respective sound recording itself, for each of the plurality of sound recordings. This may be useful for identifying one or more outliers among the individual rate estimates, by indicating where the rate estimates of all of the other sound recordings are in close agreement.

In a set of embodiments, the method comprises a further step of using at least the aggregate rate estimate to calculate a respective decision metric for each sound recording, and using the decision metric to determine whether to assign the respective individual rate estimate or a different rate estimate to the sound recording. In this way, the aggregate rate estimate may be used to inform whether to keep the individual rate estimate for the sound recording, or replace it with what is deemed to be a more reliable estimate.

The decision metric for a sound recording may be calculated as, or in dependence on, an amount by which the individual rate estimate for the sound recording differs from the aggregate rate estimate. The aggregate rate estimate here may be a weighted combination of all of the plurality of individual rate estimates. In this case, a large decision metric would indicate a larger difference between the individual rate estimate and the average rate estimate, and therefore would indicate a higher likelihood of unreliability as an outlier. The decision metric may be defined such that it increases when the individual rate estimate is more likely to be unreliable.

The decision metric for each sound recording may be compared to a threshold value (which may be constant across all the sound recordings), and a rate estimate may be assigned to the sound recording in dependence on said comparison. If the decision metric exceeds the threshold value, the individual rate estimate may not be assigned to the respective sound recording and a different rate estimate may be assigned to the sound recording instead. In some embodiments, the different rate estimate may be the individual rate estimate with a highest quality measure across all of the sound recordings. Alternatively, the different rate estimate may be the aggregate rate estimate (e.g. determined over all the sound recordings, or over all the sound recordings other than the respective sound recording), or a non-weighted average of the individual rate estimates, such as the median or the mean.

In a set of embodiments, if the decision metric for a sound recording exceeds the threshold value and the quality measure of all the sound recordings is below a minimum value, an average (e.g. the median or the mode) of the individual rate estimates may be assigned to the sound recording (e.g. instead of the rate estimate from the highest quality recording).

The rate estimate assigned to each sound recording may then be used to segment the respective sound recording. For example, as discussed above, an assigned heart rate may be used to inform the process of splitting each heart beat into segments corresponding to diastole and systole phases.

In a set of embodiments, a decision metric for a sound recording is calculated using a respective confidence score for the sound recording, and/or a respective deviation score. The confidence score may depend at least in part on a confidence measure for the sound recording (e.g. as described above). The deviation score may be calculated as a function of an individual deviation value for the respective sound recording and a collective deviation value. An individual deviation value for a respective sound recording may be calculated as, or in dependence on, the difference between the individual rate estimate for the sound recording and the aggregate rate estimate. A collective deviation value for a respective sound recording may be calculated as the standard deviation of two or more of the individual rate estimates of the plurality of sound recordings. In particular, the collective deviation value for a sound recording may calculated as the standard deviation of all of the plurality of individual rate estimates apart from the individual rate estimate determined for the respective sound recording (e.g. σ₋ᵢ as described below). Such a deviation score can help in detecting outliers—i.e. individual rate estimates that may be spurious—by identifying situations where an individual rate estimate for a sound recording differs significantly from the rates estimates of the other sound recordings and where those other sound recordings are all in close agreement with each other.

The confidence score for each sound recording may be calculated in dependence on a confidence measure for the respective sound recording, and on a highest individual confidence measure out of a plurality of individual confidence measures calculated for the plurality of sound recordings. For example, the confidence score may depend on an individual confidence measure normalised by the highest confidence measure across all of the sound recordings. It may be calculated as a weighted or non-weighted linear combination of the confidence measure for the sound recording and such a relative confidence measure.

The apparatus may further comprise an electronic stethoscope for generating the plurality of sound recordings of the heart or respiratory system of the human or animal body. The electronic stethoscope may be integral with the processing system (e.g. in a common housing), or the apparatus may comprise or be configured to provide a wired or wireless connection from an output of the electronic stethoscope to an input of the processing system.

Some embodiments of the method may comprise capturing the plurality of sound recordings, e.g. as the sounds emanate from the heart or respiratory system of the human or animal body. However, in other embodiments, the plurality of sound recordings may be captured, or may have been captured, by a process that is separate from the disclosed method.

The plurality of sound recordings may be received (e.g. by a processing system as disclosed herein) from an electronic stethoscope or from a memory of the apparatus. The plurality of sound recordings may be received while the sounds are emanating from the human or animal body (e.g. being received in real-time as live sound recordings), or at a later time. In some embodiments, the plurality of sound recordings may have been captured before the commencement of the method (e.g., minutes, hours or days before the method is performed) and/or by a process that is separate from the claimed method.

The apparatus may comprise a memory for storing a result of any calculating or determining step disclosed herein, e.g. as one or more binary values. It may comprise an output, such as a display or a network connection for outputting any such result.

Each sound recording may span any number of cardiac or respiratory cycles—e.g. two, ten, a hundred or more.

The software may be provided on a non-transitory computer-readable medium.

Features of any aspect or embodiment described herein may, wherever appropriate, be applied to any other aspect or embodiment described herein. Where reference is made to different embodiments or sets of embodiments, it should be understood that these are not necessarily distinct but may overlap.

### BRIEF DESCRIPTION OF THE DRAWINGS

Certain preferred embodiments of the invention will now be described, by way of example only, with reference to the accompanying drawings, in which:

FIG. 1 shows a schematic overview of a clinician capturing a sound recording from the body of a subject using an electronic stethoscope in accordance with the present invention.

FIG. 2 shows a schematic view of a human chest indicating exemplary locations for capturing different sound recordings in accordance with the present invention.

FIG. 3 shows a high-level flow diagram of the method for processing the sound recording at each location for further analysis in accordance with the present invention

FIG. 4 is a plot showing an example of an autocorrelation function which is used to identify the heart rate estimate of each recording in accordance with the present invention.

FIG. 5 is a flow chart which gives an overview of the method for estimating the heart rate using multiple recordings in accordance with the present invention.

### DETAILED DESCRIPTION

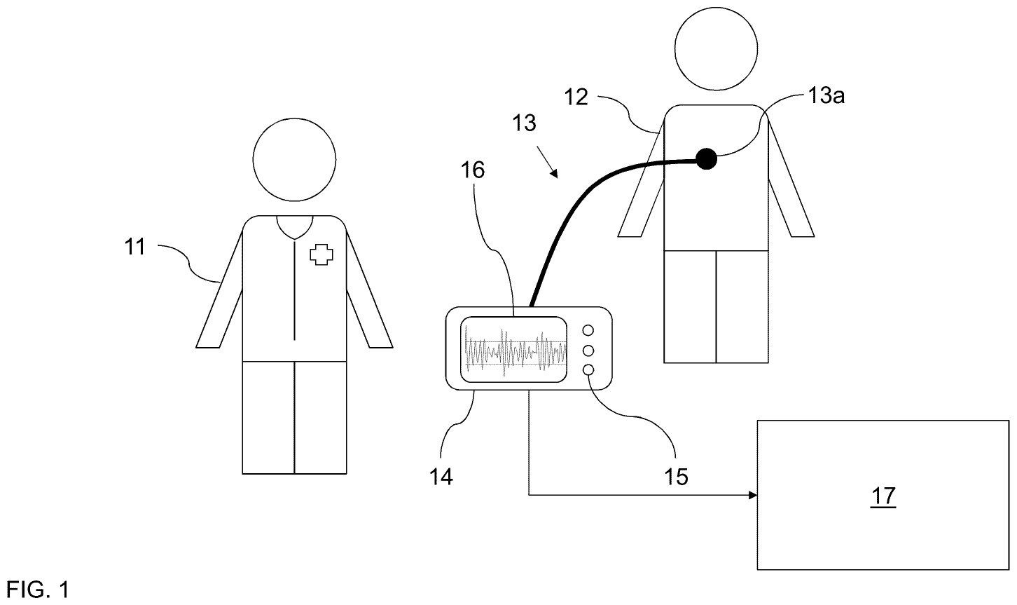

FIG. 1 shows a schematic overview of a clinician 11 capturing a sound recording from the body of a subject 12 using an electronic stethoscope 13. The clinician 11 places the chest piece 13a of the electronic stethoscope 13 at a location on the exterior of the chest of a subject 12, and controls a recording system 14 using controls 15 and a display 16 such that a recording of sound emanating from the heart, spanning a given length of time, is captured for that location. The captured sound recording can be digitised by the recording system 14 and subsequently be processed by a processing system 17. The electronic stethoscope 13 may be physically connected to the processing system 17, or it may be able to communicate by wireless link. The processing system 17 could alternatively be integral with the recording system 14.

In accordance with embodiments of the present invention, the clinician 11 captures a sound recording from each of multiple different locations on the chest of the subject 12. In some embodiments, the chest piece 13a may comprise multiple microphones and be able to capture sound recordings simultaneously at multiple locations, but in this example the clinician 11 move the chest piece 13a between locations on the chest over time, such that each sound recording is made over a different time period.

The processing system 17 (e.g. a workstation or a laptop computer) may contain a processor and a memory storing software for execution by the processor for performing some or all of the processing operations disclosed herein. It may have its own input and output interfaces, such as a display screen.

The processing system 17 can then use sound recordings captured at different locations to process each of the captured sound recordings for determining clinically useful information. In the set of embodiments described herein, the captured sound recordings are of the human heart; however, it should be understood that the methods disclosed herein may be used to process recordings that capture sound emanating from the heart, or from the respiratory system (e.g. lungs and/or windpipe), of any animal subject. They could also be used to process sound recordings captured from the same location but at different times.

It is known to capture sound recordings of the heart at different locations around the exterior of the chest. These different recordings may better inform an understanding of the behaviour of the subject's heart, with recordings at some locations providing better indicators of specific medical conditions or abnormalities than others. When a clinician is aiming to examine a patient thoroughly with a view to identifying abnormalities, it is known that there are four different locations which should be examined.

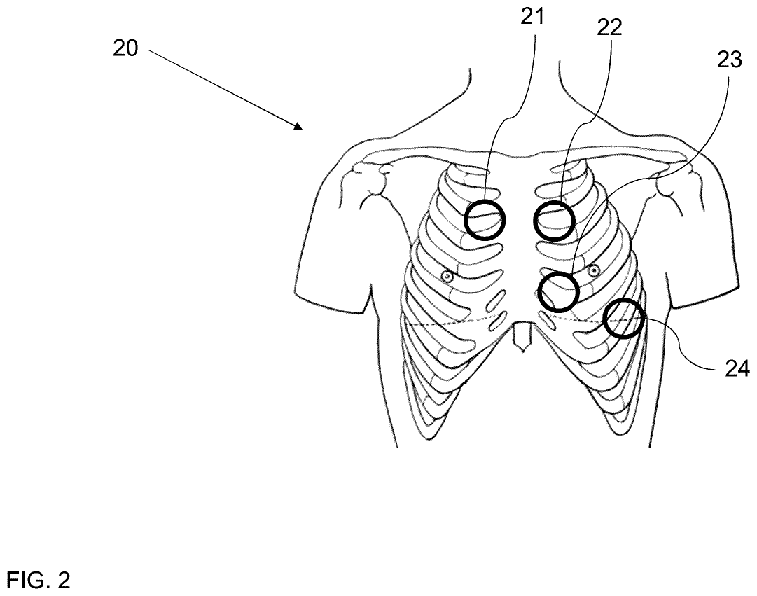

FIG. 2 shows a schematic view of a human chest 20, indicating exemplary locations 21-24 for capturing different sound recordings. These locations are adjacent the expected positions of the aortic valve (location 21), pulmonary valve (location 22), tricuspid valve (location 23) and mitral valve (location 24). In the specific example described herein, sound recordings are captured at four different locations that are known to show abnormalities, if they are present. However, in other scenarios more or fewer locations may be used.

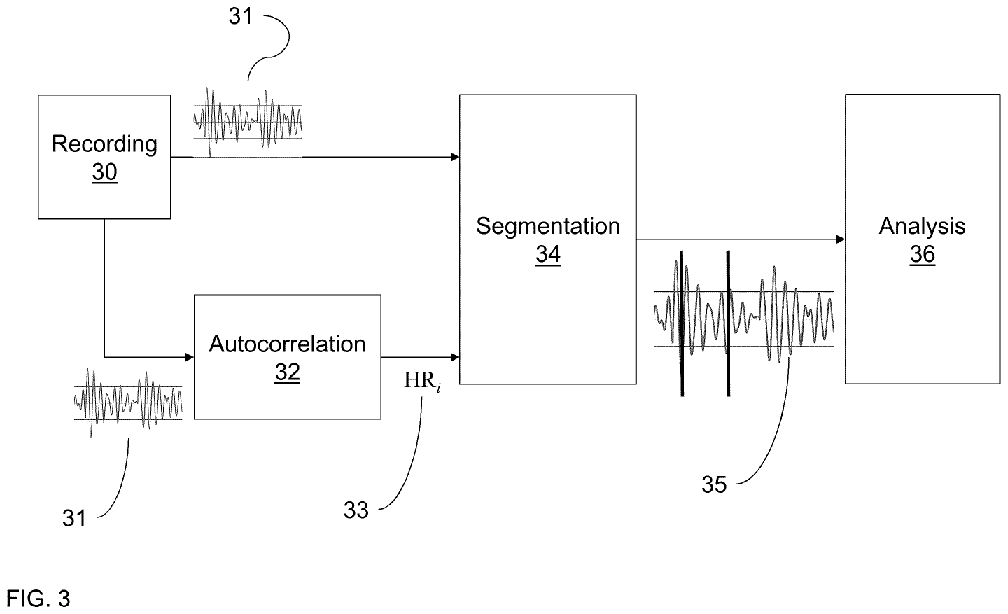

FIG. 3 shows a high-level flow diagram of the method for processing the sound recording obtained for each location for further analysis. It is often desirable to segment sound recordings of the heart so as to identify (i.e. distinguish between) systole phases and diastole phases. This segmentation then allows a clinician, computer, or artificial intelligence programme to then more straightforwardly process the recording to assess health or identify any abnormalities. The accuracy of an automated segmentation process can be improved by using a heart rate estimate to process the sound recording.

For each location (e.g. i=1, . . . , 4), a respective sound recording is performed in a recording process 30. The analog sound is digitised using an analog-to-digital converter. Each sound recording may be pre-processed in the analog and/or digital domains, and the resulting recording 31 given as input to an autocorrelation process 32. For example, in order to better reveal relevant periodicities, each sound recording may be processed by down-sampling to a rate of ˜2200 HZ, then by band-pass filtering with a 4th order Butterworth filter using corner frequencies at 25 and 400 Hz, then by taking the homomorphic envelogram, and then by removing spikes, before performing autocorrelation on the resulting signal.

The autocorrelation process 32 outputs a heart rate estimate 33, HRᵢ, for each sound recording 31. The heart rate may be represented in any of various equivalent ways—e.g. as a value in beats per minute (bpm), or as a cycle duration in milliseconds. Both the heart rate estimate 33 for each sound recording and each digital sound recording 31 are given as input to a segmentation process 34. The output of this segmentation process 34 is a set of segmented sound recordings 35 that includes data for each sound recording identifying segments of diastole and segments of systole with the recording. One or more of the segmented sound recordings 35 may be further analysed in an analysis process 36 by a clinician, computer program, or artificial intelligence.

A more accurate heart rate estimate 33 helps to generate an accurate segmented recording 35. The autocorrelation process 32 may determine an estimate of the heart rate 33 by auto-correlating the recording 31 and analysing the result to identify cross-correlation peaks. The autocorrelation may be performed by correlating (e.g. calculating the dot product of), after any pre-processing of the sound recording, a copy of the recording with varying delay against the non-delayed version. The time delay which produces the largest peak in the auto-correlation function within an appropriate search interval will typically correspond to the period of the heart rate. A rate estimate may then be determined from this time period. However, if the sound recording 31 is of poor quality (e.g. has a low signal-to-noise ratio) it can be difficult to obtain an accurate heart rate estimate for the sound recording 31, which could lead to inaccurate segmentation. Methods disclosed herein may help to mitigate this risk.

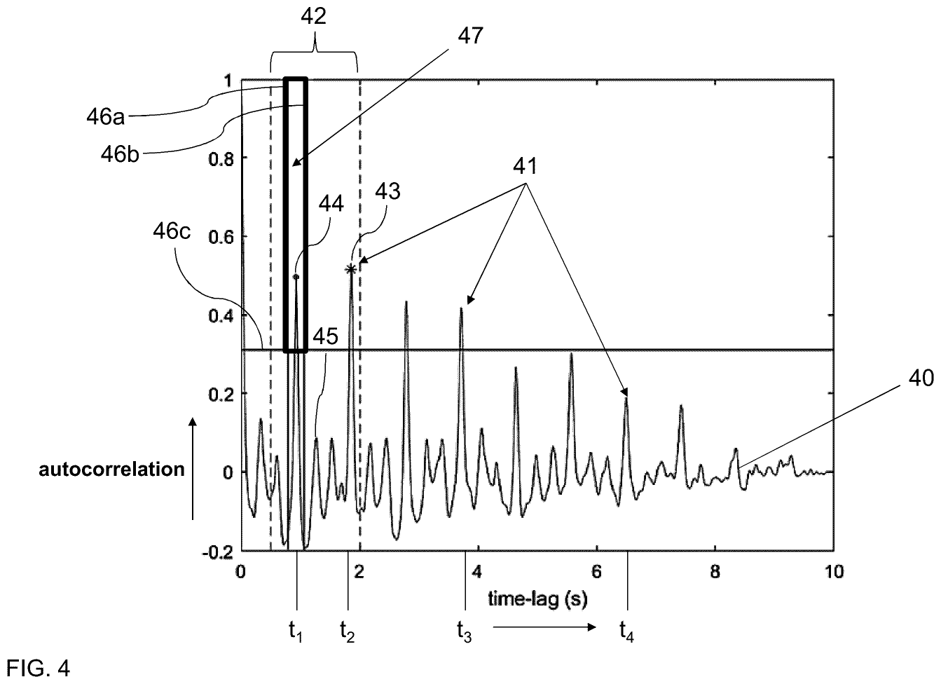

FIG. 4 is a plot showing an example of an autocorrelation function 40 which is calculated and analysed within the autocorrelation process 32 to calculate the heart rate estimate 33 for a particular sound recording 31.

The autocorrelation function 40 has several peaks 41 at various time delays, indicating similarity between the original and delayed signals at time-lags t₁, t₂, t₃ and t₄ etc. This indicates that the sound recording has some periodicity over time periods corresponding to each of the peak locations. A delay search interval 42 of 1.5s duration is shown on the plot between a time delay of 0.5s and 2s.

It is advantageous for the search interval 42 to correspond to the maximum and minimum expected period of a resting heart rate within reasonable physiological boundaries (e.g. covering most ages and conditions of subject 12). In this case, a delay of 0.5s would indicate a heart rate of 120 bpm (showing a repeating heart beat every 0.5s), whereas a 2s delay would indicate 30 bpm.

If processing the autocorrelation plot shown in FIG. 4 in a naïve manner, the largest peak 43 within the search interval, indicated at t₂ in the example shown, might be used to determine the heart rate estimate. However, the presence of interference or noise in the sound recording may produce spurious peaks in the auto correlation function 40, and therefore affect the accuracy of a rate estimate. The applicant has appreciated that the rate estimate may be determined more reliably by applying further processing steps to the autocorrelation function, rather than just selecting the largest auto-correlation peak. The applicant has therefore devised enhancements to this naïve approach which allow for a more robust heart rate estimate calculation. These enhancements may be implemented within the autocorrelation process 32.

The first enhancement accounts for situations in which the delay time of the largest peak of the heart rate auto-correlation corresponds to an integer multiple of the delay time of another peak in the search interval 42. This instance is depicted in FIG. 3, where the true period of heart cycle is t₁, but the largest peak is at delay t₂. Selecting the largest peak 43 at t₂ in this case would result in the heart rate estimate being wrong by a factor of two, because both t₁, the true heart rate delay period, and t₂, twice the length of the true delay, have been captured within the search interval 42.

In order to prevent this erroneous rate estimate from being generated, instead of simply selecting the tallest peak 43, a plurality of candidate peaks (e.g. three peaks) is selected first. In the exemplary plot shown in FIG. 3, the three highest peaks 43, 44 and 45 of the autocorrelation function in the search interval are identified, forming the set of candidate peaks. The maximum peak 43 is identified—in this example, at the time t₂. In order to search for smaller delay periods that are close to a unit fraction (e.g. a half or a third) of the maximum peak 43's delay period t₂, threshold values 46a, 46b, 46c are calculated from the position of the maximum peak (*) 43. The thresholds specify a minimum time threshold 46a, a maximum time threshold 46b and a minimum autocorrelation threshold 46b. Together with an inherent maximum bound on the autocorrelation of +1, these thresholds define a rectangular unit-fraction search region 47. The threshold values are calculated such that a peak that is at least 60% of the magnitude of the highest peak 43, and that has a time delay close to half that of the highest peak 43, will fall within the unit-fraction search region 47.

The exemplary unit-fraction search region 47 shown in FIG. 4 is a half-period search region and occupies the time-lag interval/x and the autocorrelation interval I_{y} defined by:

$${I}_{x} = [ 0.85 \cdot \frac{{x}^{*}}{2} , 1.15 \cdot \frac{{x}^{*}}{2} ]$$

$${I}_{y} = [ 0.6 \cdot {y}^{*} , ∞ ]$$

where (x*, y*) are the time and autocorrelation coordinates for the primary maximum peak 43. Calculating intervals as defined above enables the autocorrelation process 32 to identify a smaller, but still significant peak at half of the delay period of the primary peak 43 (with a 15% time tolerance either side). Where such a peak exists, it is more likely to indicate the true heart rate, with the highest peak corresponding to a time offset of two heartbeats. This is because a time offset of half of one heartbeat will not typically exhibit strong autocorrelation and so would not meet the minimum autocorrelation threshold 46b.

If another peak falls inside the unit-fraction search region 47, as shown to be the case in FIG. 4, then it is used to estimate the heart rate instead of the maximum-peak 43. In this case, the candidate peak 44 at t₁ falls within unit-fraction search region 47, so is taken as the true delay period for the heart rate estimate of the given recording.

Thus, the first enhancement provides a more robust estimate of the heart rate for each individual recording. The accuracy of the estimates may then be further improved by considering the heart rate estimates which are determined from the other locations.

The sound recordings captured at the four different locations may vary considerably in terms of how strongly periodic they are. This consequently affects the reliability of both the individual heart rate estimates obtained from the autocorrelation function for each recording, and the eventual segmentation applied to the recordings. The applicant has appreciated that the average heart rate of individuals typically varies only slightly between the recordings obtained for the four different locations if performed sequentially within a reasonably short period of time. Therefore, by using the sound recordings captured at different locations to check the variation in heart rate estimates across the four locations, the reliability of each individual heart rate estimate may be improved.

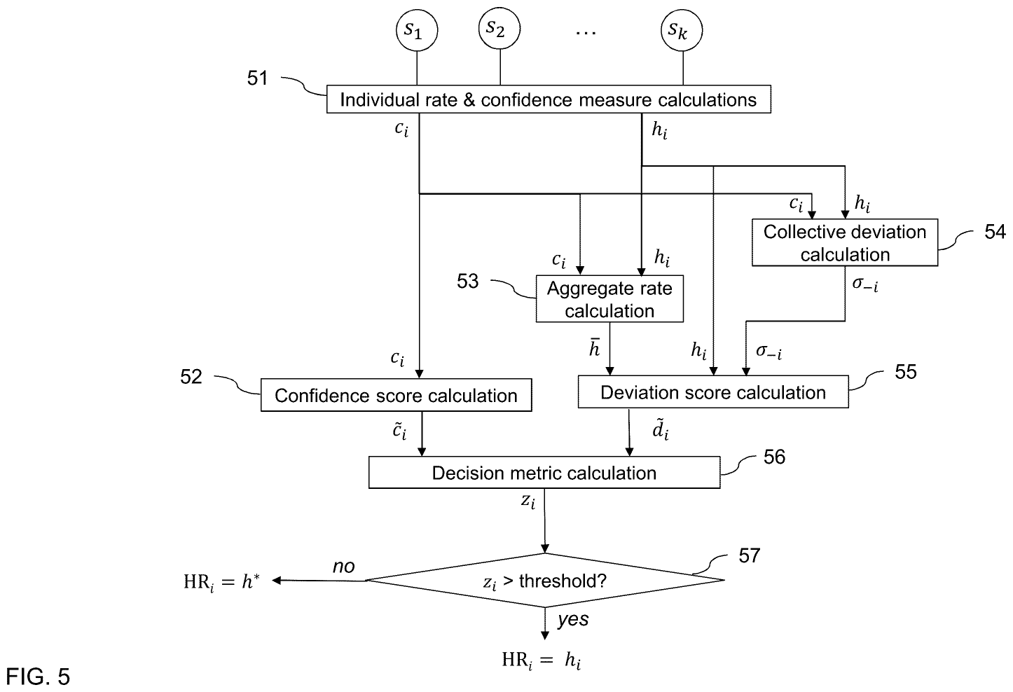

FIG. 5 is a flow chart which gives an overview of a method for estimating heart rate using multiple sound recordings s₁, . . . , sₖ in accordance with an embodiment of the present invention. In some embodiments, there are four sound recordings (i.e. k=4) from the four respective locations on the chest (referred to as locations i=1, . . . , k), but there could be more or fewer in some examples. Some or all of the processes described below may be implemented by an autocorrelation module as part of the autocorrelation process 32 described above.

An individual heart rate & confidence measure calculation process 51 receives the sound recordings and calculates a respective individual heart rate estimate hᵢ and an associated confidence measure cᵢ are calculated for each location, i=1, . . . , k using an autocorrelation function (ACF) determined for each captured sound recording sᵢ. It may determine the heart rates using an autocorrelation method as described above with reference to FIG. 3, and the confidence measures as described below.

The confidence measures cᵢ are provided to a confidence score calculation process 52 and to an aggregate heart rate calculation process 53.

The heart rate estimates hᵢ are provided to the aggregate heart rate calculation process 53 and also to a collective deviation calculation process 54 and a deviation score calculation process 55. The deviation score calculation process 55 also receives an aggregate heart rate estimate h̅ (representing an average of heart rates across the locations) from the aggregate heart rate calculation process 53, and a collective deviation value σ₋ᵢ (representing the spread of heart rate estimates across the set of recordings) from the collective deviation calculation process 54.

In the confidence-score calculation process 52, a confidence score \tilde{c}ᵢ is calculated for each location, i, from the confidence measures cᵢ, which depend on features of the heart rate peak and the autocorrelation function. Details of how these may be calculated are given below. Each confidence score \tilde{c}ᵢ is calculated such that it reflects the likelihood that the heart rate estimate from the individual heart rate & confidence measure calculation process 51 is reliable, with a higher confidence score indicating greater reliability.

In the deviation-score evaluation process 55, a deviation score \tilde{d}ᵢ is calculated for each location, i, based on the degree of variation of the heart rate estimate hᵢ at the respective location, i, from the heart rate estimates h, at the other locations, j≠i. This deviation score is calculated such that it reflects the likelihood that the heart rate estimate h, is an outlier, with a higher deviation score indicating a greater chance that the estimate is erroneous (e.g. based on an autocorrelation peak having an offset that is not equal to the actual cardiac cycle length).

A decision metric calculation process 56 receives the confidence scores \tilde{c}ᵢ and deviation scores \tilde{d}ᵢ from the confidence score calculation process 52 and the deviation-score calculation process 55, and calculates a decision metric zᵢ for each location, using a combination of the respective confidence score and deviation score.

The decision metrics zᵢ are provided to a heart-rate assignment process 57, which determines whether the respective individual heart rate estimate hᵢ for each sound recording sᵢ should be kept or discarded in favour of assigning a sound recording an alternative heart rate estimate h* that has a higher confidence score. This more confident heart rate estimate may be the heart rate estimate hᵢ of one of the other sound recordings captured at a different location j≠i such as the heart rate estimate that has the highest confidence score. The decision is taken such that the individual heart rate estimate hᵢ will be replaced if the confidence score \tilde{c}ᵢ is low and the deviation score \tilde{d}ᵢ is high.

The individual heart rate & confidence measure calculation process 51 and the confidence-score calculation process 52 cooperate to calculate the confidence score či for each recording location i based on some or all of the following three properties of the auto correlation function (ACF) for the sound recording sᵢ:
1. peak prominence, measured by S_{prominence}. This measures how pronounced the main heart rate peak is relative to the adjacent valleys either side of it. A less prominent peak indicates less certainty in its associated time delay.
2. periodicity, represented by a calculated quantity S_{periodicity}. This measures how well the ACF can be approximated with a periodic function. It can be expected that, due to the periodic nature of a sequence of heart beats, ACF peaks will appear at integer multiples of the delay period that corresponds to the true heart rate. Irregularity in the spacing of the peaks of the ACF may therefore indicate that they are caused by other sources of sound not emanating from the heart, e.g. captured as noise.
3. height of the heart rate peak relative to the other peaks in the search interval, represented by calculated quantity S_{rel.height}. If the heart rate peak is not clearly distinguished from other peaks which may be caused by noise, this indicates a higher degree of uncertainty over the validity of the peak.

The processes used to calculate the three quantities S_{prominence}, S_{periodicity}, S_{rel.height}, and are explained in further detail below.

### Peak Prominence

Peak prominence S_{prominence} is a local measure of peak prominence, and reflects the distinctiveness of a peak relative to its local neighbourhood. This is achieved by locating the adjacent valleys, one on the left, and one on the right, (see the MATLAB documentation, for example, for well-known techniques that can be applied to do this), and computes the height of the peak relative to the highest of the two neighbouring valleys. The applicant has found that S_{prominence} captures information which is especially useful for distinguishing between high and low quality autocorrelations in the present context. The other two parameters described below may further inform a more meaningful confidence score. However, some embodiments may use only S_{prominence}, or only S_{prominence} and S_{periodicity} (i.e. not using S_{rel.height}).

### Periodicity

In order to quantify an estimate for periodicity of the auto-correlation function (i.e. the regularity of primary peaks over a wider time interval spanning several heart beats), several signal processing steps may be applied.

For example, first, a proportion of the auto-correlation function may be discarded, on account of it not providing much information. As an optional further step, the slow changing trend of the ACF can be captured with a smoothing function (e.g., a Gaussian kernel, using the MATLAB function ‘smoothdata’) using a wide kernel window (e.g. of width 4.57s, although this width may be tuned as appropriate such that the slow-changing trend is adequately captured). The output of the smoothing function, M_{trend}, is then subtracted from the ACF to form a de-meaned ACF r₀.

A discrete cosine transformation of r₀ is then computed, and a subset of the cosine coefficients are discarded, thus obtaining a periodic function G_{periodic} which approximates r₀. Taking the four largest coefficients of the cosine transformation coefficients has been found to produce the desired results, although a number greater or smaller than four may be retained in other embodiments. Finally, periodicity is scored based on how well G_{periodic} approximates r₀ relative to a baseline fit provided by the smoothing function M_{trend}, with fit measured in terms of a cost function, for example, by calculating the root-mean-squared-error (RMSE):

$${S}_{periodicity} = 1 - \frac{RMSE ( {G}_{periodic} - {r}_{0} )}{RMSE ( {M}_{trend} - {r}_{0} )}$$

### Relative Peak Height

Relative peak height S_{rel.height} measures the prominence of the main heart rate peak relative to the height of the second highest peak in the autocorrelation search interval. This metric measures peak prominence in a more global sense by calculating the ratio

$$\frac{{y}_{HR}}{{y}^{′}} ,$$

where y_{HR} is the height of the main heart rate peak, and y′ is the height of the tallest peak within heart rate search interval that is not the main heart rate peak.

A confidence measure c; for each individual sound recording may be computed according to the general formula:

$${c}_{i} = {g}_{1} ( {S}_{prominence} ) * {g}_{2} ( {S}_{periodicity} ) * {g}_{3} ( {S}_{rel . height} )$$

where g₁, g₂ and g₃ are non-decreasing functions, for example gⱼ(x)=ex, gⱼ(x)=x², gⱼ(x)=x, or gⱼ(x)=1, in any combination of functions.

Setting gⱼ(x)=1 enables only one or two of the measures to be used, rather than all three.

For example, the confidence measure c; may be computed according to:

$${c}_{i} = {S}_{prominence} \cdot {S}_{periodicity} \cdot ( {α}_{1} \cdot sigmoid ( {S}_{rel . height} - {μ}_{1} ) + {μ}_{2} )$$

However, in some variant embodiments, it could be calculated as:

$${S}_{prominence} \cdot {S}_{periodicity}$$

or just

For ease of interpretation in these and the following equations, the αᵢ denote positive tuning parameters, while tuning parameters that can take both negative and positive values are denoted by μᵢ. These parameters may be set to appropriate values, e.g. through trial and improvement on test data, to tune the performance of the processes. As an example, in some embodiments the tuning parameters may be set as below:

$${α}_{1} = 1.5 , {μ}_{1} = 1.4 , {μ}_{2} = 0.9$$

To capture relative confidence (i.e. how one sound recording compares with the others) as well as this absolute confidence measure, cᵢ, for a sound recording i, a confidence score \tilde{c}ᵢ is calculated by the confidence-score calculation process 52 as follows, where c* denotes the highest (i.e. maximum) confidence score across all locations:

$${\tilde{c}}_{i} = {α}_{2} \cdot {( \frac{{c}_{i}}{{c}^{*}} )}^{{α}_{3}} + {α}_{4} \cdot {c}_{i}$$

where, for example:

$${α}_{2} = - 0.22 , {α}_{3} = 1.5 , {α}_{4} = - 0.08$$

This definition means that having a high relative confidence

$$\frac{{c}_{i}}{{c}^{*}}$$

and a high absolute confidence measure cᵢ will each contribute to a sound recording sᵢ getting a high confidence score \tilde{c}ᵢ and so decrease the tendency for the heart rate estimate for location i to be discarded.

The deviation score \tilde{d}ᵢ for each recording location i is calculated based in part on the variance amongst the heart rate estimates from the different recordings as explained below. The deviation score \tilde{d}ᵢ also depends in part on the absolute deviation of the individual heart rate estimate from a weighted average of the estimates from the different locations (i.e. an aggregate estimate).

First, an aggregate heart rate estimate h̅ is calculated by the aggregate rate calculation process 53 as a weighted sum of the heart rate estimates from all k (e.g. all four) locations i, as given below, where hᵢ denotes the individual heart rate estimate for location i, which is weighted by a respective quality measure, qᵢ:

$$\overline{h} = {\sum }_{i = 1}^{k} ( {h}_{i} \cdot {q}_{i} )$$

The quality measure for each sound recording may be calculated as:

$${q}_{i} = \frac{{C}_{i}}{{\sum }_{j = 1}^{k} {C}_{j}}$$

An individual deviation value dᵢ for each location can then be calculated within aggregate rate calculation process 53, using this aggregate heart rate estimate, as dᵢ=|hᵢ−h̅|.

A collective deviation value σ₋ᵢ representative of the degree of spread within the other heart rate estimates (i.e. those for the locations other than location i) is also calculated, by the collective deviation calculation process 54, to capture the degree of variation or agreement within the set of other estimates, as follows:

$${σ}_{- i} = 1 / 2 \cdot \sqrt{{\sum }_{j \neq i} {( {h}_{j} - {\overline{h}}_{- i} )}^{2}}$$

where h̅₋ᵢ is an average of the k−1 (e.g. three) heart rate estimates excluding hᵢ, which may be a weighted average calculated using the same quality-measure weights qᵢ as used for h̅, but in this example is calculated as a non-weighted mean average.

The full weighted average h and/or any of the self-excluded weighted averages h̅₋ᵢ may be considered a form of “aggregate rate estimate” as disclosed and claimed herein.

In this example, a standard deviation is used for calculating σ₋ᵢ, but other embodiments could use any appropriate measure of collective spread of the heart rate estimates to calculate the collective deviation value.

To capture the variation between heart-rate estimates both for the individual sound recording from location i, and collectively amongst the heart-rate estimates, a deviation score is calculated for each location by the deviation-score calculation process 55 as \tilde{d}ᵢ:

$${\tilde{d}}_{i} = {( \frac{{d}_{i}}{1 + {α}_{5} \cdot sigmoid ( {α}_{6} \cdot {σ}_{- i} )} )}^{{α}_{7}}$$

where:

$${α}_{5} = 1.4 , {α}_{6} = 0.25 , {α}_{7} = 1.5$$

The form of denominator above is purely exemplary; it may take any form such that high consensus amongst the other locations decreases the tendency of location i to stick with its own estimate.

A decision metric z; is then calculated for each location i by the decision-metric calculation process 56 as a weighted combination of the confidence score \tilde{c}ᵢ and the deviation score \tilde{d}ᵢ for that location:

$${z}_{i} = {α}_{8} \cdot {\tilde{d}}_{i} - {α}_{9} \cdot {\tilde{c}}_{i}$$

where:

$${α}_{8} = 0.09 , {α}_{9} = 1$$

The decision metric zᵢ is thus defined in such a way that it increases (i.e. making it more likely that the location's heart-rate estimate hᵢ will be discarded) with increasing individual deviation dᵢ from the average estimate h, and with increasing agreement amongst the other locations (as measured by 1/σ₋ᵢ), and with a decreasing relative confidence score či.

In this way, the individual heart rate estimates are either kept or discarded for each sound recording depending on whether the confidence score for the respective sound recording is below a threshold, and on whether it deviates strongly from the estimates of the other locations.

If the decision variable zᵢ exceeds a set threshold, then the autocorrelation process 32 discards the heart rate estimate hᵢ and replaces it with h*, the estimate with the highest confidence score from across all the other k−1 (e.g. three) locations, as the final heart rate estimate HR, for location i; otherwise it keeps its current estimate and outputs HRᵢ=hᵢ to the segmentation process 34. Alternatively, in some embodiments, the aggregate heart rate h could be used to replace a discarded individual rate estimate hᵢ. In still further embodiments, any of the other heart rate estimates deemed to be more accurate could be used. In the case that all locations have low confidence scores (i.e. below a fixed threshold), the median of the heart rate estimates h; may be assigned to the sound recordings for all k locations.

This second enhancement to the naïve approach introduced above uses recordings from across the different positions to inform the processing of each individual recording and therefore provides a more robust estimate of heart rate.

These improved heart rate estimates may be useful in their own right (e.g. by being output to a human operator, or provided to another process), but they find particular utility in enabling accurate segmentation of the four sound recordings, as described above.

Although the processing has been described with reference to four sound recordings, it will be appreciated that it may be applied to any number of sound recordings and/or locations (e.g. two, three, four, five or more sound recordings).

It will be appreciated by those skilled in the art that the invention has been illustrated by describing one or more specific embodiments thereof, but is not limited to these embodiments; many variations and modifications are possible, within the scope of the accompanying claims.

1-17. (canceled)

18. An apparatus for determining rate estimates from sounds emanating from a heart or respiratory system of a human or animal body, wherein the apparatus comprises a processing system and a memory and is configured to:
receive a plurality of sound recordings of a heart or respiratory system of a human or animal body, wherein each sound recording is or has been captured by a microphone positioned at a respective location on the exterior of the human or animal body;
for each of the plurality of sound recordings, determine a respective individual rate estimate for the sound recording by analysing a respective autocorrelation function of the sound recording and determining a respective quality measure for the sound recording; and
determine an aggregate rate estimate for the heart or respiratory system by evaluating a weighted combination of two or more of the plurality of individual rate estimates, wherein each of the individual rate estimates included in the weighted combination is weighted at least in part by the respective quality measure for the respective sound recording.

19. The apparatus of claim 18, wherein determining the respective quality measure comprises determining a prominence of a primary peak in the respective autocorrelation function relative to an adjacent region of the autocorrelation function.

20. The apparatus of claim 18, wherein determining the respective quality measure comprises determining a prominence of a primary peak relative to one or more further peaks of the autocorrelation function.

21. The apparatus of claim 18, wherein determining the respective quality measure comprises determining a periodicity of a succession of primary peaks of the autocorrelation function.

22. The apparatus of claim 18, wherein the respective quality measure equals or depends on a ratio of a respective confidence measure for the respective sound recording to the sum of respective confidence measures for all of the plurality of sound recordings.

23. The apparatus of claim 18, wherein the processing system is further configured, for each sound recording, to:
use the aggregate rate estimate to calculate a respective decision metric for the sound recording;
compare the decision metric to a threshold value; and
assign either the respective individual rate estimate or a different rate estimate to the sound recording in dependence on the comparison.

24. The apparatus of claim 23, wherein the processing system is further configured to use the rate estimate assigned to each sound recording to segment the respective sound recording.

25. The apparatus of claim 23, wherein the processing system is further configured to calculate the decision metric for each sound recording using a respective confidence score for the sound recording, wherein the respective confidence score is calculated using an individual confidence measure for the respective sound recording normalised by a highest individual confidence measure out of a respective plurality of individual confidence measures calculated for the plurality of sound recordings.

26. The apparatus of claim 23, wherein the processing system is further configured to calculate the decision metric for each sound recording using a respective deviation score, wherein the respective deviation score is calculated as a function of an individual deviation value, wherein the respective individual deviation value is calculated as, or in dependence on, the difference between the individual rate estimate for the sound recording and the aggregate rate estimate.

27. The apparatus of claim 23, wherein the processing system is further configured to calculate the decision metric for each sound recording using a respective deviation score, wherein the respective deviation score is calculated as a function of a collective deviation value for the respective sound recording, wherein the respective collective deviation value is calculated as the standard deviation of all of the plurality of individual rate estimates apart from the individual rate estimate determined for the respective sound recording.

28. The apparatus of claim 18, wherein determining each individual rate estimate comprises:
identifying a primary autocorrelation peak from a set of one or more candidate peaks in the respective autocorrelation function; and
calculating the individual rate estimate from a time delay of the primary autocorrelation peak.

29. The apparatus of claim 28, wherein determining each individual rate estimate comprises:
determining a unit-fraction search interval defining a time interval which spans or is centred around a predetermined unit fraction of the time delay of the identified primary autocorrelation peak;
determining whether another of the set of candidate peaks, in addition to the identified primary autocorrelation peak, falls within the unit-fraction search interval and has an autocorrelation above a minimum level; and
where such another peak is identified, using the other peak to determine the respective individual rate estimate, instead of the identified primary autocorrelation peak.

30. The apparatus of claim 18, further comprising an electronic stethoscope for generating the plurality of sound recordings of the heart or respiratory system of the human or animal body.

31. A method of determining rate estimates from sounds emanating from a heart or respiratory system of a human or animal body, wherein the method is performed by a processing system and comprises:
receiving a plurality of sound recordings of a heart or respiratory system of a human or animal body, wherein each sound recording is or has been captured by a microphone positioned at a respective location on the exterior of the human or animal body;
for each of the plurality of sound recordings, determining a respective individual rate estimate for the sound recording by analysing a respective autocorrelation function of the sound recording and determining a respective quality measure for the sound recording; and
determining an aggregate rate estimate for the heart or respiratory system by evaluating a weighted combination of two or more of the plurality of individual rate estimates, wherein each of the individual rate estimates included in the weighted combination is weighted at least in part by the respective quality measure for the respective sound recording.

32. The method of claim 31, wherein each sound recording is or has been captured by a microphone positioned at a different respective location on the exterior of the human or animal body.

33. The method of claim 32, wherein the plurality of sound recordings are four sound recordings of the heart, wherein each sound recording is or has been captured adjacent a different respective one of an aortic valve, a pulmonary valve, a tricuspid valve, and a mitral valve of the heart.

34. The method of claim 31, wherein determining the respective quality measure comprises determining a periodicity of a succession of primary peaks of the autocorrelation function.

35. The method of claim 31, wherein the respective quality measure equals or depends on a ratio of a respective confidence measure for the respective sound recording to the sum of respective confidence measures for all of the plurality of sound recordings.

36. The method of claim 31, comprising, for each sound recording:
using the aggregate rate estimate to calculate a respective decision metric for the sound recording;
comparing the decision metric to a threshold value; and
assigning either the respective individual rate estimate or a different rate estimate to the sound recording in dependence on the comparison.

37. A non-transitory computer-readable storage medium storing instructions which, when executed on a processing system, cause the processing system to perform the method of claim 31.
